(19) United States
(12) Patent Application Publication
(10) Pub. No.: US 2014/0316180 A1
(43) Pub. Date: Oct. 23, 2014

(54) APPARATUSES AND METHODS FOR HYDRODYNAMIC CAVITATION TREATMENT OF LIQUIDS

(71) Applicant: QUANTUM VORTEX, INC., State College, PA (US)

(72) Inventor: Max I. FOMITCHEV-ZAMILOV, State College, PA (US)

(73) Assignee: Quantum Vortex, Inc., State College, PA (US)

(21) Appl. No.: 13/869,017

(22) Filed: Apr. 23, 2013

(51) Int. Cl. B01J 19/00; C07C 4/02; C10G 15/08

(52) U.S. Cl. CPC B01J 19/008; C10G 15/08; C07C 4/02
USPC 585/752; 208/125; 422/127

(57) ABSTRACT

An apparatus for breaking molecular bonds of a liquid may include a first arrangement configured to create macroscopic flow of a liquid such that a molecule of the liquid has a velocity corresponding to a bond disassociation energy of the molecule. The apparatus may also include a second arrangement configured to collide the macroscopic flow of liquid with an obstacle. The collision results in molecular collisions having an energy that exceeds the bond disassociation energy of the molecule.

### TECHNICAL FIELD

The present disclosure relates generally to apparatuses and methods for hydrodynamic cavitation treatment of liquids. More particularly, the disclosure relates to apparatuses and methods for hydrodynamic cavitation treatment of hydrocarbons in the oil and gas industry.

### BACKGROUND

Cavitational treatment of liquid hydrocarbon such as crude oil, fuel oil, bitumen, and various biofuels is known to reduce their viscosity and increase the yield of light fraction extractable via subsequent atmospheric and/or vacuum distillation. Such treatment of hydrocarbons (which is not limited to cavitation) with the objective to increase their quality is generally referred to as upgrading.

The upgrading due to cavitation becomes economically viable and commercially attractive if the following three necessary conditions are met: 1) the process must produce energy densities that are high enough to brake molecular bonds and create free radicals; 2) when recombining the radicals must form new chemical species with the desired properties, which were deficient in the original mix; 3) the energy or capital costs must be competitive compared to the established upgrading methods such as thermal, catalytic, or hydrocracking.

Fortunately, hydrodynamic cavitation reactors satisfy these conditions. When powered by 15-200 kW electric motors these devices can generate acoustic energy density in excess of 1 MW/m², which is sufficiently high to break long hydrocarbon chains and upgrade crude. However, there is still the problem of the radical recombination of the hydrocarbon chains.

Although the technique of cavitational oil cracking has been known in the Soviet Union since the early sixties, the technology is virtually unknown in the West, and there are only a few small companies in Russia and Ukraine that develop, manufacture, and export the cavitation equipment mostly to customers in China, India, Spain, and Brazil. The U.S. petroleum industry and the American economy too stand to benefit from industrial applications of cavitation to hydrocarbon processing and reap substantial economical benefits such as energy savings, reduced fuel costs, and cleaner emissions.

Some conventional apparatuses exist with the capabilities for heating a liquid as a result of the unavoidable or concomitant mechanical effects on it of such forces as, specifically, the forces of friction during contact with a surrounding environment, the forces of internal friction during agitation of a stream of liquid, and the forces arising during hydraulic impacts and cavitation. The energy that is expended during these processes on heating a liquid is viewed as a natural energy loss.

The effect of heating a liquid as a result of the deliberate—though this may not be the primary purpose—effects on it of mechanical vibrations in the sonic or ultrasonic range is also widely known in technology. And in this particular case the energy that is expended in heating the liquid is traditionally viewed as unavoidable energy losses. Particularly well-known from the current state of technology (V. I. Bigler et al., “The Dispersal Of Various Materials In A Device Of The Hydraulic Siren Type,” in the collection of scientific studies No. 90 of the Moscow Institute for Steel and Alloys “Application Of Ultrasonic Waves In Metallurgy,” the “Metallurgiya” Publishing House, 1977, p. 73-76) is the effect of rapid heating of a liquid utilizing a device of the so-called hydrodynamic siren type. This device contains a rotating wheel having a cavity with a feeding or conveying aperture for supplying the liquid and a series of outlet apertures that are uniformly distributed along the periphery and that are installed in its peripheral wall with a conical external surface, and a stator having a cavity with an outlet aperture for expelling the liquid and a series of inlet apertures that are uniformly distributed along the periphery and that are installed in its wall, which latter is adjacent at a small distance to the peripheral wall of the rotating wheel, in which both the series of apertures of the rotating wheel and the series of apertures of the stator are arranged on a plane of the revolution [of the wheel]. When the wheel is rotating, the liquid flowing out from the outlet apertures of the rotating wheel and toward the inlet aperture of the stator is subject to the effect of induced mechanical vibrations of a defined frequency, depending upon the rate of revolution of the rotating wheel and upon the number of its outlet apertures. In the given case, the activation of these vibrations in the liquid is only designed to disperse the material that is contained in the liquid. Nonetheless, the authors noted the fact of an abnormally rapid heating of the liquid. They explained this rapid heating by an increase in the hydraulic resistance during the run-over or overflow of the liquid from the cavity of the rotating wheel into the cavity of the stator. In the case at hand, however, the authors did not provide an explanation for this phenomenon in purely quantitative terms.

Also well-known at the current level of technology (European Patent Publication No. EP 0 673 677 A1—incorporated herein by reference) is a method of heating a liquid by means of processing it by means of mechanical vibrations. This method involves the injection of the liquid to be processed into the cavity of a revolving rotating wheel; bringing the liquid to revolve along with the rotating wheel; the expulsion of the liquid from the cavity of the rotating wheel through a series of outlet apertures on its peripheral cylindrical surface; the injection of the liquid into a cavity of the stator through at least one inlet aperture in the concentric surface of the stator lying as close as possible to the peripheral cylindrical surface of the rotating wheel; during which there occur periodic abrupt interruptions in the flow of the liquid that serve to activate or stimulate mechanical vibrations in the liquid. As a result of such processing, the liquid that is arriving in the cavity of the stator, as established by the authors, is heated to a higher degree than could be explained by the aggregate or overall hydraulic losses. However, this effect of an abnormal heating of a liquid, which in principle was merely detected, was neither sufficient not stable enough to be used in practical applications with assured success. The reason for that may reside in the incorrect selection of the parameters of the process, and specifically in the incorrect selection of the rate of revolution of the rotating wheel and of its interdependence upon the geometrical dimensions and the number of the outlet apertures of the rotating wheel.

Taking into account the preceding, an alternative method and device for heating a liquid was devised (U.S. Pat. No. 6,227,193 B1—incorporated herein by reference). The method in accord with this patent application includes supplying a liquid to be processed into the cavity of a revolving rotating wheel; the bringing of the liquid undergoing processing to revolve along with the rotating wheel; the expulsion of the liquid from the cavity of the rotating wheel through a series of outlet apertures on its peripheral cylindrical surface; the injection of the liquid into the cavity of the stator through at least one inlet aperture on the concentric surface of the stator lying as close as possible to the peripheral cylindrical surface of the rotating wheel; during which there occurs periodic abrupt interruptions in the flow of the liquid that stimulate mechanical vibrations in it. According to available information, this represented the first time an attempt was made to express the preferred dependence between the linear velocity of the liquid on the periphery of a specified radius and this radius in mathematical terms.

The device for implementing the described method for heating a liquid contains a rotor, including a shaft located in bearings; a rotating wheel that is connected to the shaft and made in the form of a disk with a peripheral annular wall having cylindrical exterior and interior surfaces in which are located a series of outlet apertures for passing the liquid, which outlet apertures are uniformly arranged along the periphery; a stator that contains the rotating wheel as it revolves or spins and that has an inlet aperture for feeding in the liquid and an outlet aperture for expelling the liquid, and two concentric walls that from both sides come as close as possible to the peripheral annular wall of the rotating wheel; in both concentric walls of the stator are located at least one aperture for the passing of the liquid, which aperture is lying on a plane with the positioning of the series of apertures of the rotating wheel.

Thus there exist such chemical-engineering processes that feature the activation energy within 100 and 400 kJ/mole, and over. To intensify such energy-consuming processes an ultrasonic radiation having an intensity of or above 1 MW/m² is required. It is only in this case that an ultrasonic processing becomes economically justifiable.

Further, it is well known that hydrocarbon cracking (i.e., breaking of the C—C molecular bond) occurs when molecules collide and the energy of collision exceeds that energy of the C—C bond itself. For a heavy hydrocarbon such as, for example, C₇₂H₁₄₀, having a molar mass of about 1000 g/mol, cracking of the hydrocarbon molecule occurs at a temperature greater than or equal to 400° C. The Maxwell distribution can be used to compute the average thermal velocity for the hydrocarbon molecule at 400° C. The average thermal velocity of the hydrocarbon molecule, which is sufficient to create collision energy of the hydrocarbon molecules that exceeds the bond energy of the C—C bond, is about 100 m/s.

However, to take advantage of the aforementioned hydrocarbon cracking properties of heavy hydrocarbon such as, for example, C₇₂H₁₄₀, the hydrocarbon liquid would have to be heated to 400° C. Unfortunately, the cost associated with the equipment for heating the hydrocarbon liquid to 400° C. and the energy cost for the heating process significantly reduce the economic benefits of hydrocarbon cracking.

Because of potential importance of the applications of hydrocarbon cracking in oil and gas industry, it is desirable to produce improved hydrodynamic cavitation devices and methods that can effectuate hydrocarbon cracking without the costs associated with heating a hydrocarbon liquid.

### SUMMARY OF THE INVENTION

In some aspects of the disclosure, an apparatus for breaking molecular bonds of a liquid may include a first arrangement configured to create macroscopic flow of a liquid such that a molecule of the liquid has a velocity corresponding to a bond disassociation energy of the molecule. The apparatus may also include a second arrangement configured to collide the macroscopic flow of liquid with an obstacle. The collision results in molecular collisions having an energy that exceeds the bond disassociation energy of the molecule.

According to various aspects of the disclosure, an apparatus for conditioning a hydrocarbon liquid may include a source of liquid containing a hydrocarbon molecule at a desired cracking temperature. A first arrangement may be configured to create macroscopic flow of the liquid such that the hydrocarbon molecule has a velocity comparable with a thermal velocity of the hydrocarbon molecule at the desired cracking temperature. A second arrangement may be configured to collide the macroscopic flow of the liquid with an obstacle, and the collision results in molecular collisions having an energy that exceeds a disassociation energy of a C—C bond of the hydrocarbon molecule.

In accordance with some aspects of the disclosure, a method for breaking molecular bonds of a liquid may include creating macroscopic flow of a liquid such that a molecule of the liquid has a velocity corresponding to a bond disassociation energy of the molecule, and colliding the macroscopic flow of the liquid with an obstacle. The collision results in molecular collisions having an energy that exceeds the bond disassociation energy of the molecule.

In various aspects of the disclosure, a method for conditioning a hydrocarbon liquid may include supplying a liquid containing a hydrocarbon molecule at a desired cracking temperature, creating macroscopic flow of the liquid such that the hydrocarbon molecule has a velocity comparable with a thermal velocity of the hydrocarbon molecule at the desired cracking temperature, and colliding the macroscopic flow of the liquid with an obstacle. The collision results in molecular collisions having an energy that exceeds a disassociation energy of a C—C bond of the hydrocarbon molecule.

According to some aspects of the disclosure, a method for conditioning a hydrocarbon liquid may include introducing a hydrocarbon liquid into a cavity of a wheel rotatably coupled with a stator. The wheel includes a peripheral annular surface having a plurality of outlet openings spaced equidistantly along the circumference of the annular surface. The openings have a width in the circumferential direction that is 10-50% of the width of the spacing between adjacent openings. The stator has a peripheral annular surface concentric with the annular surface of the wheel. The stator has a plurality of radially-extending grooves at the interior surface thereof. The grooves are spaced equidistantly along the circumference of the annular surface of the stator. The grooves have a width in the circumferential direction that is substantially equal to the spacing between adjacent grooves. The method includes rotating the wheel relative to the stator to force the hydrocarbon liquid into the outlet openings at the peripheral annular surface of the wheel. The wheel is rotated at a rate sufficient to create sonic vibrations in the hydrocarbon liquid within the openings. The energy of the sonic vibration within a first opening is released when the first opening is aligned with a first one of the grooves. The energy of the sonic vibration is accumulated within a second opening when the second opening is not aligned with one of the grooves. The method includes discharging the hydrocarbon liquid from the grooves via an annular channel in fluid communication with the grooves.

The present disclosure describes a hydrodynamic cavitation apparatus and method that rapidly rotates a perforated rotor to generate acoustic excitation to achieve the requisite acoustic energy densities on the order of 1-10 MW/m². According to the disclosure, the apparatus and method produces high density of acoustic energy over a wide surface area (i.e. around the rotor) thus producing much larger cavitation volume and higher energy density when compared to the traditional piezoelectric transducer or sonotrode-based devices.

The hydrocarbon/chemical activation process and apparatus relies on extreme sheering in non-Newtonian liquids that occurs in narrow rotor-stator space, ionization/radicalization due to high electrostatic/electromagnetic fields, and/or molecular activation due to magnetic bonding (formation of Santilli's magnecular states). These effects leave to chemical reaction acceleration and breaking (cracking) of long hydrocarbon chains. The cracking can occur later due to metastable nature of the magnecular states. Therefore the so activated crude oil may not crack immediately but will crack with time or when heated.

In some aspects, the disclosure is directed to apparatuses and methods for hydrodynamic cavitation treatment of hydrocarbons having such a construction arrangement and operation that will be instrumental in increasing the intensity of acoustic energy to 1-10 MW/m². This colossal energy stimulates profuse cavitation, confined to slots of the rotor. The massive sonic energy forms plasma within the bubbles, the bubbles form Marx generator-like discharges, which further contribute to molecular radicalization and hydrocarbon cracking. To prevent recombination of radicals and reduce the formation of aromatics the addition of hydrogen or hydrogen donor is required to the processed mixture. Fortunately, the addition of gasses also stimulates cavitation thus further intensifying the process. Therefore, the combination of all these factors makes efficient cavitation-induced hydrocarbon cracking feasible (at least in principle) and thus potentially economically important.

### BRIEF DESCRIPTION OF THE DRAWINGS

FIG. 1 is a schematic view of an exemplary apparatus in accordance with various aspects of the disclosure;

FIG. 2 is a longitudinal axial section of an exemplary apparatus in accordance with various aspects of the disclosure;

FIG. 3 is a partial cross section of the annular chamber of the exemplary apparatus of FIG. 2; and

FIG. 4 is a front end view of the exemplary apparatus of FIG. 2 with the stator cover removed.

### DETAILED DESCRIPTION

Reference will now be made in detail to specific embodiments or features, examples of which are illustrated in the accompanying drawings. Generally, corresponding or similar reference numbers will be used, when possible, throughout the drawings to refer to the same or corresponding parts.

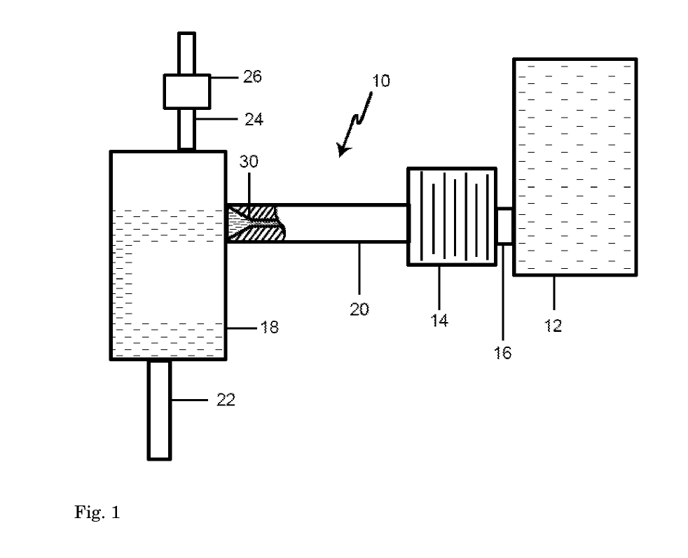

FIG. 1 illustrates an exemplary apparatus for cracking liquid molecules at ambient temperatures (i.e., without necessarily heating the liquid). The apparatus 10 includes a source of liquid, such as tank 12. The tank 12 may be configured to maintain the liquid, which in some aspects may comprise a heavy hydrocarbon, substantially at ambient temperature.

The apparatus 10 may include a pump 14 in fluid communication with the tank 12 via a first flow line 16. The apparatus 10 may also include a chamber 18 in fluid communication with the pump via a second flow line 20 terminating with a nozzle 30 at the chamber 18. The pump 14 may be operable to create, generate, and/or facilitate fluid flow from the tank 12 to the chamber 18. The second flow line 20 has an orifice diameter and nozzle 30 selected to direct the liquid into the chamber at a desired flow velocity. The cracked liquid exits the chamber 18 through the drain 22. The evaporated light hydrocarbons and incondensable gases exit the chamber 18 through the vent 24 controlled by the valve 26.

In accordance with various aspects, the pump 14, the second flow line 20, and/or the nozzle 30 create macroscopic flow of liquid from the tank 12 to the chamber 18 such that the liquid has a desired flow velocity. The desired flow velocity is a velocity of the liquid that, when colliding with an obstacle, converts the impact into internal energy corresponding to a bond disassociation energy of a molecule of the liquid. The obstacle may be a barrier, such as, for example, a wall of the chamber 18 or some other physical structure. In some aspects, the obstacle may be another flow of the liquid, for example, another macroscopic flow of the liquid.

In one exemplary embodiment, the tank 12 may contain a hydrocarbon liquid, such as C₇₂H₁₄₀, for example, substantially at ambient temperature. As described, supra, disassociation of the C—C bonds of a C₇₂H₁₄₀ molecule occurs at a temperature of 400° C., where the average thermal velocity of the molecule is 100 m/s. However, in apparatus 10, the hydrocarbon liquid is stored in tank 12 substantially at ambient temperature. Rather than heating the hydrocarbon liquid, the liquid in the tank 12 is pressurized to about 100 bars of pressure so that the average thermal velocity for molecules of the pressured liquid is 100 m/s. The tank 12 may be pressurized by any conventional means. The pump 14, the second flow line 20, and/or the nozzle 30 create and/or facilitate macroscopic flow of the pressurized hydrocarbon liquid from the tank 12 to the chamber 18 such that the liquid has a flow velocity in excess of 100 m/s. The hydrocarbon liquid entering the chamber 18 collides with an obstacle, such as, for example, another macroscopic flow of hydrocarbon liquid or a barrier such as a wall of the chamber 18 or other structure (not shown). The collision between the macroscopic flow of the pressurized hydrocarbon liquid at 100 m/s with the obstacle results in molecular collisions having an impact energy that is converted to an internal energy of the hydrocarbon molecules that exceeds the bond disassociation energy of the C—C bond of the hydrocarbon molecules.

It should be appreciated that the pump 14 and the second flow line 20 having a desired orifice and nozzle may be replaced by a rotor-stator machine (not shown). For example, a rotor-stator machine can be constructed with a 1 m diameter wheel. Operating such a device at 60 Hz will cause the wheel to reach a radial velocity of 100 m/s. Thus, if the pressurized hydrocarbon fluid (100 bars) of the previous example is supplied to the rotor-stator machine, macroscopic flow of the liquid in the rotor-stator gap will be colliding with the rotor and stator walls at 100 m/s. These collisions at 100 m/s result in molecular collisions having an impact energy that is converted to an internal energy of the hydrocarbon molecules that exceeds the bond disassociation energy of the C—C bond of the hydrocarbon molecules.

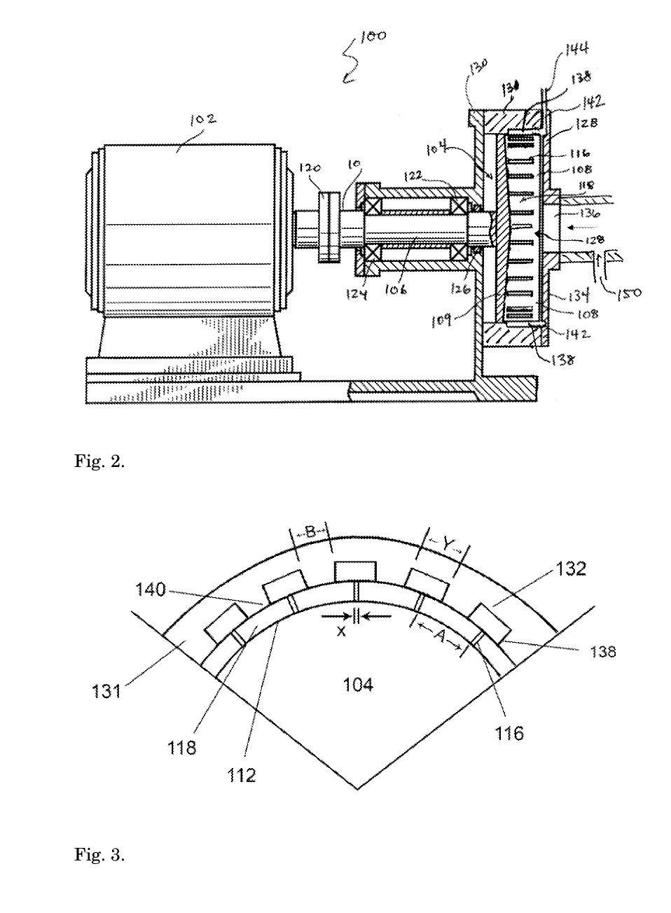

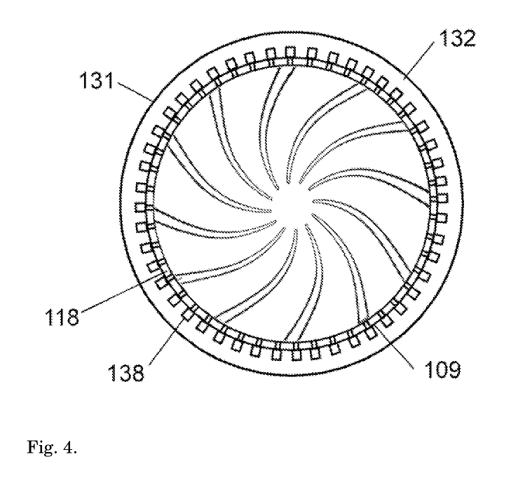

FIGS. 2-4 illustrate an exemplary apparatus for hydrodynamic cavitation treatment of hydrocarbons. The apparatus 100 includes a motor 102, for example, an electric motor, controllable to operate at a desired rate of revolution. The apparatus further includes a rotor 104 having a drive shaft 106 connected with a rotating wheel 108. According to some aspects, the rotor 104 may include an impeller 109 such that the rotor 104 may operate as a centrifugal pump, which has the rotating wheel 108 rigidly fixed at the outlet thereof. The rotating wheel 108 may be made integral with the impeller 109 or, alternatively, the rotating wheel 108 and impeller 109 may be separable elements that are fixedly coupled together. In some aspects, the impeller 109 can be removed from the apparatus and replaced with an external pump capable of generating high static pressures (1-100 bars).

The rotating wheel 108 may be formed as a disk 110 having a peripheral annular wall 112. The annular wall 112 has a cylindrical external surface 114. The wall 112 includes a series of outlet openings 116. According to various aspects, the outlet openings 116 may be uniformly distributed along the peripheral annular wall 112. In some embodiments, the width X of the openings 116 is small relative to the width A of blank regions 118 that separate the openings 116 circumferentially about the annular wall 112. For example, the width A of blank regions 118 of the annular wall 112 of the rotor 104 is equal to about 1-10 times the width of the openings 116.

The width of the blank needs to be sufficiently large to allow enough time for the liquid in the rotor opening to compress under the action of the static pressure produced by the impeller or by the external pump. The time necessary to achieve the compression can be calculated as radius of the rotor (or the length of the rotor channel if it is not negligible) divided by the speed of sound in the liquid.

The rotor 104 is coupled with the motor 102 via a coupling 120 so that the rotor 102 operably rotates the rotor 104, drive shaft 106, and wheel 108. The drive shaft 106 is supported by bearings 122, 124 and is equipped with a gasket 126 to prevent leakage of liquid toward the drive shaft 106 and bearings 122, 124.

The apparatus includes a stator housing 130 surrounding a stator 131 having a peripheral annular wall 132 concentric with the annular wall 112 of the rotor 104. The annular wall 132 includes an inner surface 133. The annular wall 112 of the rotor 104 and the annular wall 132 of the stator 131 are sized such that, when assembled, the inner surface 133 of the stator 131 and the external surface 114 of the rotor 104 are sufficiently close together to prevent any meaningful flow of liquid therebetween. For example, the surfaces 114, 133 are spaced just far enough apart so that no friction is generated between the rotor 104 and stator 131 as they relatively rotate. The stator housing 130 includes a removable cover member 134 that can be removed from the remainder of the stator housing 130 in order to provide access to the stator 131 and the rotating wheel 108 of the rotor 104. The cover 134 includes an inlet aperture 136 for receiving a supply of the liquid for processing.

The rotor 104 includes a cavity 128 delimited by the disk 110 and the annular wall 112 of the rotating wheel 108, and the cover member 134 of the stator housing 130. The cavity 128 receives, via the inlet aperture 136, liquid that will undergo processing as discussed in more detail below. Upstream of the inlet aperture 136 is a line 150 for introducing a dispersing gas in the form of micron-sized (e.g., <100 microns) gas bubbles into the flow of liquid prior to the liquid entering the cavity 128. According to various aspects, the dispersing may be hydrogen, carbon dioxide, methane, methanol, air, or naphtha. The micron-sized gas bubbles may range in size from 1-10 microns, and in some aspects from 2-4 microns. The size of the gas bubbles may be determined in accordance with methodologies known in the art.

The annular wall 132 of the stator 131 includes a series of grooves 138 cut therein. The cover member 134 includes an outlet channel 142 in fluid communication with one or more of the grooves 138. According to various aspects, the grooves 138 may be uniformly distributed along the peripheral annular wall 132. In some embodiments, the width Y of the grooves 138 is substantially the same as the width B of blank regions 140 that separate the grooves 138 circumferentially about the annular wall 132 (FIG. 3). For example, the width B of blank regions 118 of the annular wall 132 of the stator 131 is equal to about 1-2 times the width Y of the grooves 138.

In some aspects, the width X of the openings 116 in the annular wall 112 of the rotor 104 is 0.1-0.5 the width Y of the grooves 138 in the annular wall 132 of the stator 131.

The apparatus 100 may include a circulation line 144 in fluid communication with the outlet channel 142. The circulation line 144 can be arranged to direct all or a portion of the treated liquid into repeated processing through the apparatus. It should be appreciated that the circulation line 144 may be provided with one or more valves (not shown) for directing fluid flow. For example, crude oil may be repeatedly pumped through the apparatus 100 under 2-5 MPa pressure and with addition of up to 3% by volume of dispersing gas.

The outside surface of the stator housing 130 may be coated with a layer of a sound- and heat-insulating material and/or protected with a metal casing in order to reduce energy losses into the surrounding environment. It should be appreciated that when a large amount of a liquid medium is to be processed, the overall dimensions of the working cavity can be increased as needed.

In operation, liquid being processed is fed to the cavity 128 via inlet aperture 136. Upstream of the aperture 136, the liquid is infused with a dispersing gas in the form of micro-sized bubbles that contains hydrogen ions. The electric AC motor 102 drives the shaft 106, which rotates impellor 109 and rotor 104. The liquid passes onto the impeller 109 rotating on the drive shaft 106. Thus, the liquid acquires some kinetic energy on the impeller 109, which is partly converted into the energy of elastic vibrations of the liquid when the liquid passes through periodically aligned and shut openings 116 and grooves 138 in the annular walls 112, 132 of the rotor 104 and stator 131, respectively. In order to make an efficient use of the acoustic/sound energy generated by the rotor—stator combination, it is necessary to maintain an optimum static pressure, which is determined by specific physical properties of the liquid under processing. The liquid processing time depends on the period of time within which the liquid passes throughout the device's working cycle. The processing time can be extended for part or all of the liquid under processing by re-circulating the liquid any desired number of times through the device's working cycle with the aid of the circulation line 144 interconnecting the device's outlet pipe connector to the inlet pipe connector 16 thereof. The recycle ratio is adjusted by the circulation line 144, which is equipped with valves and/or other flow restrictions mechanisms. After having been processed in the apparatus the liquid is discharged through the outlet channel 142.

The apparatus is essentially a centrifugal pump where the processed liquid is accelerated by a rapidly rotating perforated rotor 104 and rotating wheel 108 and then forced by the impellor 109 through the openings 116 in the rotor 104. Thus, a significant portion of the liquid is trapped in the openings. As such, large pressures are built in the liquid, which contribute to cracking of the large hydrocarbon chains of, for example, crude oil. After the large hydrocarbon chains are cracked, the hydrogen ions introduced into the liquid upstream of the cavity 128 will bond to the cracked hydrocarbon chains, thereby prevented recombination of the undesirable large chains.

In an exemplary operation, the apparatus 100 can be supplied with a liquid from, for example, a tank similar to that shown in FIG. 1. The tank may contain a hydrocarbon liquid, for example, C₇₂H₁₄₀, at ambient temperature but at a boosted pressure of about 100 bars. The apparatus 100 can be operated to create, generate and/or facilitate a 100 m/s flow rate of the hydrocarbon liquid through the apparatus 100, for example, via inlet aperture 126, into cavity 128, and radially toward outlet openings 116 of the rotating wheel 108 of the rotor 104. The flow of liquid will be regularly colliding with the stator wall 132 when the openings 116 are closed, thereby creating hydraulic shocks. These collisions at 100 m/s result in molecular collisions having an impact energy that is converted to an internal energy of the hydrocarbon molecules that exceeds the bond disassociation energy of the C—C bond of the hydrocarbon molecules.

It should be appreciated that the temperature of the hydrocarbon liquid in the tank can be increased to 200° C. Such heating would still be quite energy efficient, and therefore cost efficient, compared with heating to 400° C. At 200° C., the thermal velocity of the molecules is 88 m/s. Thus, the apparatus 100 can be operated to create, generate and/or facilitate an 88 m/s flow rate of the hydrocarbon liquid through the apparatus 100, for example, via inlet aperture 126, into cavity 128, and radially toward outlet openings 116 of the rotating wheel 108 of the rotor 104. The flow of liquid will be regularly colliding with the stator wall 132 when the openings 116 are closed, thereby creating hydraulic shocks. These collisions at 88 m/s result in molecular collisions having an impact energy that is converted to an internal energy of the heated hydrocarbon molecules that exceeds the bond disassociation energy of the C—C bond of the heated hydrocarbon molecules.

The apparatus generates acoustic waves when the liquid exits through the slots in the rotors. In such configuration, each slot can be viewed as a Helmholtz resonator forming a chain capable of accumulating large acoustic energy. The so-trapped acoustic energy stimulates powerful cavitation that in turn causes chemical disassociation/radicalization of molecules. While ionization of vapors (e.g. the creation of plasma) inside collapsing bubbles will create a momentary magnetic field one can reasonably expect no net effect due to random orientation of the transient magnetic fields caused by the multitude of bubbles. However, the actual distribution of bubbles may not be random due to stable vortices pinned in the rotor's slots. Due to cavitation these vortices will be full of streaming bubbles. If each individual bubble is viewed as a microscopic capacitor where the charged ‘plates’ are formed by ionized gasses, the bubble vortex becomes analogous to a multi-stage Marx generator where the breakdown of dielectric in between the bubbles will result in massive discharges with voltages easily reaching into MV range. Assuming modest polarization energy of 1 eV, it is estimated that the bubble growth during the expansion phase will result in voltage build-up up to 10 kV per bubble. Consequently it takes only 100 closely packed bubbles forming a multi-stage Marx generator-like discharge to reach the voltages on the order of 1 MV, which no doubt assists molecular ionization/radicalization and contributes to the increased efficiency of the present apparatus when compared to conventional sonotrode-based ultrasonic activators.

Thus, apparatuses and methods according to the disclosure are capable of highly efficient transformation of mechanical energy into acoustic energy with density on the order of 1-10 MW/m². This colossal energy stimulates profuse cavitation, confined to slots of the rotor. The massive sonic energy forms plasma within the bubbles, the bubbles form Marx generator-like discharges, which further contribute to molecular radicalization and hydrocarbon cracking. To prevent recombination of radicals and reduce the formation of aromatics the addition of hydrogen or methane is required to the processed mixture. Fortunately, the addition of gasses also stimulates cavitation thus further intensifying the process.

The apparatus 100 has the ability to generate enormous sonic energy densities on the order of 1-10 MW/m² by virtue of both acoustically and hydrodynamically-induced cavitation. Furthermore, the circulation line 144 allows repeated processing of the same liquid to maximize the cracking effect.

The present invention can find application in diverse branches of industry for performing various chemical-engineering processes based on use of the effect of acoustic energy on a substance and on the nature of physico-chemical processes performed.

From the foregoing, it will be appreciated that, although specific embodiments have been described herein for purposes of illustration, various modifications or variations may be made without deviating from the spirit or scope of inventive features claimed herein. Other embodiments will be apparent to those skilled in the art from consideration of the specification and figures and practice of the arrangements disclosed herein. It is intended that the specification and disclosed examples be considered as exemplary only, with a true inventive scope and spirit being indicated by the following claims and their equivalents.

What is claimed is:

1. An apparatus for breaking molecular bonds of a liquid, comprising:
a first arrangement configured to create macroscopic flow of a liquid such that a molecule of said liquid has a velocity corresponding to a bond disassociation energy of said molecule; and
a second arrangement configured to collide said macroscopic flow of said liquid with an obstacle, said collision resulting in molecular collisions having an energy that exceeds said bond disassociation energy of said molecule.

2. The apparatus of claim 2, wherein the apparatus is configured to increase the pressure and/or temperature of the liquid so as to lower the flow velocity required to correspond to a bond disassociation energy of said molecule

3. The apparatus of claim 1, further comprising:
a source of liquid containing a hydrocarbon molecule at a desired cracking temperature, the first arrangement being configured to create macroscopic flow of said liquid such that the hydrocarbon molecule has a velocity comparable with a thermal velocity of the hydrocarbon molecule at said desired cracking temperature, said collision resulting in molecular collisions having an energy that exceeds a disassociation energy of a C—C bond of said hydrocarbon molecule.

4. The apparatus of claim 3, wherein the apparatus is configured to increase the pressure and/or temperature of the hydrocarbon liquid so as to lower the velocity of macroscopic flow of said liquid that creates molecular collisions having an energy that exceeds a disassociation energy of a C—C bond of said hydrocarbon molecule.

5. The apparatus of claim 1, further comprising a chamber providing said obstacle with which the macroscopic flow of liquid is collided.

6. The apparatus of claim 5, wherein the obstacle comprises another macroscopic flow of said liquid and/or a physical barrier.

7. A method for breaking molecular bonds of a liquid, comprising:
creating macroscopic flow of a liquid such that a molecule of said liquid has a velocity corresponding to a bond disassociation energy of said molecule; and
colliding said macroscopic flow of said liquid with an obstacle, said collision resulting in molecular collisions having an energy that exceeds said bond disassociation energy of said molecule.

8. The method of claim 7, further comprising:
increasing the pressure and/or temperature of the liquid so as to lower the flow velocity required to correspond to a bond disassociation energy of said molecule

9. The method of claim 7, comprising:
supplying a liquid containing a hydrocarbon molecule at a desired cracking temperature, said creating step creating macroscopic flow of said liquid such that the hydrocarbon molecule has a velocity comparable with a thermal velocity of the hydrocarbon molecule at said desired cracking temperature, said collision resulting in molecular collisions having an energy that exceeds a disassociation energy of a C—C bond of said hydrocarbon molecule.

10. The method of claim 9, further comprising:
increasing the pressure and/or temperature of the hydrocarbon liquid so as to lower the velocity of macroscopic flow of said liquid that creates molecular collisions having an energy that exceeds a disassociation energy of a C—C bond of said hydrocarbon molecule.

11. The method of claim 7, wherein said colliding step includes colliding the macroscopic flow of liquid with another macroscopic flow of said liquid and/oror a physical barrier.

12. The method of claim 7, further comprising:
introducing a dispersing gas into the hydrocarbon liquid prior to said supplying step.

13. The method of claim 12, wherein the dispersing gas includes hydrogen ions, and wherein the hydrogen ions of the dispersing gas bind to a molecule formed by the disassociation of the C—C bond to prevent recombination of the C—C bond of the hydrocarbon molecule.

14. A method for conditioning a hydrocarbon liquid, the method comprising:
introducing a hydrocarbon liquid into a cavity of a wheel rotatably coupled with a stator, the wheel including a peripheral annular surface having a plurality of outlet openings spaced equidistantly along the circumference of the annular surface, the openings having a width in the circumferential direction that is 10-50% of the width of the spacing between adjacent openings, the stator having a peripheral annular surface concentric with the annular surface of the wheel, the status having a plurality of radially-extending grooves at the interior surface thereof, the grooves being spaced equidistantly along the circumference of the annular surface of the stator, the grooves having a width in the circumferential direction that is substantially equal to the spacing between adjacent grooves;
rotating the wheel relative to the stator to force the hydrocarbon liquid into the outlet openings at the peripheral annular surface of the wheel, the wheel being rotated at a rate sufficient to create sonic vibrations in the hydrocarbon liquid within the openings, the energy of the sonic vibration within a first opening being released when the first opening is aligned with a first one of said grooves, the energy of the sonic vibration being accumulated within a second opening when the second opening is not aligned with one of said grooves; and
discharging the hydrocarbon liquid from the grooves via an annular channel in fluid communication with said grooves.

15. The e method of claim 14, further comprising:
introducing a dispersing gas into the hydrocarbon liquid before the hydrocarbon liquid is introduced into said cavity.

16. The method of claim 15, wherein said energy being released during the rotating step is sufficient to break longer molecular chains of the hydrocarbon liquid into shorter molecular chains.

17. The method of claim 16, wherein the dispersing gas includes hydrogen ions, and wherein the hydrogen ions of the dispersing gas bind to the shorter molecular chains to prevent recombination of the longer molecular chains.
